(19) United States
(12) Patent Application Publication
(10) Pub. No.: US 2006/0269449 A1
(43) Pub. Date: Nov. 30, 2006

(54) SUGAR CHAIN EXCISION APPARATUS

(75) Inventors: Takafumi Nakamura, Kyoto-shi (JP); Yusuke Osaka, Kyoto-shi (JP); Kazuaki Kakehi, Kyoto-shi (JP)

Correspondence Address: RANKIN, HILL, PORTER & CLARK LLP, 4080 ERIE STREET, WILLOUGHBY, OH, 44094-7836, US

(73) Assignee: SHIMADZU CORPORATION, Kyoto-shi (JP)

(21) Appl. No.: 11/420,343

(22) Filed: May 25, 2006

(30) Foreign Application Priority Data
May 30, 2005 (JP) 2005-157407

(51) Int. Cl. B32B 27/04; G01N 33/00

(52) U.S. Cl. 422/81; 436/90

(57) ABSTRACT

A reaction tank 15 for heating a sample solution containing glycoprotein in an alkali solution to separate sugar chains, and two cartridges 17a and 17b filled with ion exchange resin are provided, and the sample solution transported from the reaction tank 15 is subjected to neutralization treatment by passing the sample solution through the cartridges. The cartridges 17a and 17b are connected in parallel by two flow channel switching valves 16a and 16b. By switching the valves 16a and 16b, selection of a cartridge to be used for the neutralization treatment is made, and at the same time, acid and water are passed through the cartridge which is not in use, in order to allow regeneration of the ion exchange resin in the cartridge.

### TECHNICAL FIELD

The present disclosure relates to an apparatus for excising sugar chains from complex carbohydrates such as glycoprotein, glycolipid, proteoglycan and the like.

### RELATED ART

More than a half of proteins in the living body are subjected to modification with sugar chain after translation, and it has been recently found that such modification with sugar chain plays an important role in the expression of protein functions. Thus, sugar chains are attracting interest as an important biomolecule, together with nucleic acids and proteins, and the structural analysis or functional analysis of sugar chains are in progress, in parallel with the post-genome research focused on the structure or function of proteins.

When an analysis of the structure or function of sugar chains in complex carbohydrates such as glycoprotein, glycolipid, proteoglycan and the like is to be performed, it is necessary to first excise sugar chains from the complex carbohydrates. For such method of excising sugar chains, there has been known a method of separating sugar chains from protein or the like by reacting under heating the complex carbohydrate in an alkali solution, and an apparatus for automatically performing such sugar chain excision treatment is being developed.

[Patent Document 1] Japanese Examined Patent Application Publication No. 7-11513

When excision of sugar chains is carried out as such by mixing with an alkali solution under heating, it is necessary to perform a neutralization treatment of the sample solution after the reaction. Thus, in a related-art sugar chain excision apparatus described above, the neutralization treatment has been carried out by transporting an acid solution and an alkali solution respectively by a pump, and mixing the solutions with the sample solution after the excision reaction. However, it is difficult to control the neutralization reaction by adjusting the respective transported amounts of the acid solution and the alkali solution, and the neutralization treatment may not be properly carried out.

### SUMMARY

The disclosure below describes a sugar chain excision apparatus which can carry out a simple and stable neutralization treatment for a sample solution after performing a sugar chain excision reaction using alkali.

An example implementation of the invention is described below. A sugar chain excision apparatus for excising sugar chains from complex carbohydrate, includes:

a) a sugar chain excising section for mixing a sample solution containing complex carbohydrate with an alkali solution under heating to excise sugar chains from the complex carbohydrate in the sample solution,

b) a neutralizing section for neutralizing the sample solution after the excision reaction by the sugar chain excising section, the neutralizing section including a cartridge filled with ion exchange resin, and

c) a cartridge regenerating section for regenerating the ion exchange resin in the cartridge by passing acid and water through the cartridge.

In the sugar chain excision apparatus, the neutralizing section may include a plurality of cartridges filled with ion exchange resin, and further, it may include a cartridge selecting section for switching between a cartridge to be used for neutralization treatment and a cartridge to be regenerated. In this case, the switching of cartridges by the cartridge selecting section is preferably carried out automatically, using a threshold value for the preliminarily set cartridge use time, a threshold value for the excision treatment unit, or a combination thereof. It is also allowed to carry out switching by the user, or switching by a timing program.

The sugar chain excision apparatus may further include a pH meter disposed at the downstream of the neutralizing section, and an error noticing section for noticing errors to the user, when the pH value, as measured with the pH meter, of the sample solution transported from the neutralizing section exceeds a predetermined threshold value. Furthermore, in addition to the error noticing section or instead of the error noticing section, the sugar chain excision apparatus may include an error responding section for discarding a sample solution without collecting, when the pH value of the sample solution transported from the neutralizing section exceeds a predetermined threshold value, and for subjecting a sample which raises an error again to the excision treatment after the switching of a cartridge to be used for neutralization by the cartridge selecting section.

The sugar chain excision apparatus may also have a sample introducing/collecting section for introducing a sample solution containing complex carbohydrate into the sugar chain excising section, as well as for collecting the sample solution after the neutralization by the neutralizing section. Such sample introducing/collecting section may have a constitution in which introduction and collection of a sample are all performed using a single needle disposed at the needle driving unit, as described in Patent Document 1 for example; however, a constitution in which a sampling needle for introducing the sample and a collecting needle for collecting the sample solution are disposed together at the needle driving unit.

Various implementations may include one or more the following advantages. For example, according to the sugar chain excision apparatus, the neutralization treatment can be carried out by passing a sample solution obtained after the sugar chain excision reaction using alkali, through a cartridge filled with ion exchange resin. Thus, it is unnecessary to control the neutralization reaction through the adjustment of the transported amounts of acid and alkali that is performed in the related-art, and the neutralization treatment can be performed simply and stably.

In the case that a plurality of cartridges and a cartridge selecting section are further included, even when the capability of one cartridge for neutralization reaches saturation, it is possible to switch the cartridge using the cartridge selecting section and to continue the neutralization treatment with another cartridge while regenerating the cartridge, thus it being possible to improve the throughput.

Furthermore, in the case of having the pH meter and the error noticing section, it is possible to confirm whether the neutralization treatment has been properly carried out, and when the neutralization treatment has not been properly carried out, the user can take an appropriate action in response, thereby allowing unnecessary consumption of the sample or time to be reduced. When the error responding section as described above is further included, the action in response is automatically taken by the apparatus for the case where the neutralization treatment has not been performed properly, and thus, the user is not required to perform complicated operations and can attempt to reduce the user's efforts significantly.

Further, in the case of including a sample introducing/collecting section, the installation costs can be reduced, and at the same time, the installation space can be diminished, as compared with the case where a section for introducing a sample solution containing complex carbohydrate into the sugar chain excising section and a section for collecting the sample solution after the neutralization by the neutralizing section.

Other features and advantages may be apparent from the following detailed description, the accompanying drawings and the claims.

### BRIEF DESCRIPTION OF THE DRAWINGS

FIG. 1 is a schematic view showing an embodiment of the sugar chain excision apparatus of the present invention.

### DETAILED DESCRIPTION

Hereinafter, the best mode for carrying out the present invention will be described with reference to Examples.

### EXAMPLE

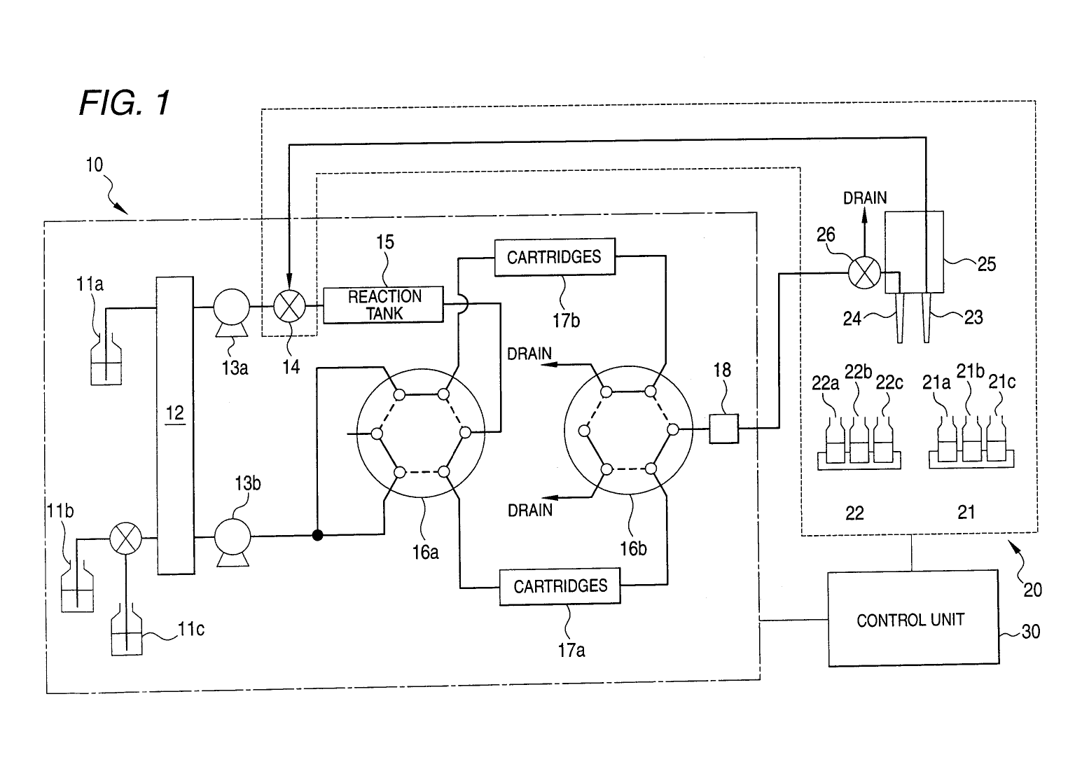

FIG. 1 illustrates a schematic constitution of the sugar chain excision apparatus of the embodiment of the present invention. The sugar chain excision apparatus includes a reaction unit 10 for performing the excision reaction or the neutralization treatment, and a sample introducing/collecting unit 20 for introducing a sample to the reaction unit 10 and for collecting the sample solution from the reaction unit 10. Operation of each unit is controlled by a control unit 30.

The reaction unit 10 includes a container 11a for holding an alkali solution for sugar chain excision, containers 11b and 11c for respectively holding an acid solution and water for cartridge regeneration, two transport pumps 13a and 13b, two flow channel switching valves 16a and 16b, a degasser 12, a reaction tank 15, two cartridges 17a and 17b filled with ion exchange resin, and a pH meter 18. The pump 13a is intended to transport the alkali solution, while the pump 13b is intended to transport acid or water. A sample inlet 14 is provided between the pump 13a and the reaction tank 15.

The flow channel switching valves 16a and 16b are all 6-port 2-position valves, and each port can take two positions such as a position to be connected as indicated by the solid line, and a position to be connected as indicated by the broken line. The cartridges 17a and 17b are connected in parallel via these flow channel switching valves 16a and 16b, and by switching the position of each flow channel switching valve, selection can be made as to which of the cartridge 17a and cartridge 17b is to be used for the neutralization and to which of the cartridges is to be used for the regeneration treatment.

The sample introducing/collecting unit 20 is an integrated element of a so-called automatic sampler and a fraction collector, and includes a sample rack 21 on which sample bottles 21a to 21c containing the sample solution before treatment are arranged, a collection rack 22 on which collection bottles 22a to 22c for collecting the sample solution after treatment are arranged, and a needle driving unit 25 comprising a sampling needle 23 for sampling of the sample solution by suction from the sample bottles and a collecting nozzle 24 for collecting the sample solution after treatment in the collection bottles. The sampling needle 23 is connected to the sample inlet 14 of the reaction unit 10, and the collecting nozzle 24 is connected to the downstream of the pH meter 18 of the reaction unit 10. Disposed between the pH meter 18 and the collecting nozzle 24 is an electromagnetic valve 26. Usually, the liquid which has passed through the pH meter 18 is set to be discharged through a drain, but in the case of collecting the sample solution after treatment, the flow channel can be switched to the side of the collecting nozzle 24.

Hereinafter, the operation of performing sugar chain excision using the sugar chain excision apparatus of the embodiment of the present invention will be described. First, the sampling needle 23 is transferred by the needle driving unit 25 to the above of a predetermined sample bottle 21a on the sample rack 21, and the sample solution is taken in by suction. The sucked-in sample is introduced to the reaction unit 10 through the sample inlet 14, mixed with an alkali solution transported by the transport pump 13a, and then sent to the reaction tank 15. The sample is heated in the reaction tank 15, and the sugar chains are excised from the complex carbohydrate contained in the sample solution.

Here, the valves 16a and 16b are both in the positions represented by the solid line, and the alkaline sample solution transported from the reaction tank 15 is sent to the cartridge 17a through the valve 16a to be neutralized. Meanwhile, to the cartridge 17b that is not used for the neutralization treatment, an acid solution and water are transported sequentially through the pump 13b and the switching valve 16a, if necessary, so as to regenerate the ion exchange resin in the cartridge 17b.

While the sugar chain excision reaction and the neutralization treatment are carried out as described above, the sampling needle 23 is rinsed with a rinsing instrument, which is not shown in the figure, and the needle driving unit 25 is moved so that the collecting nozzle 24 is positioned above the collection bottle 22a to collect the sample being treated. At this time, it is desirable that the position on the sample rack of the sample bottle 21a holding the sample being treated, is arranged to be corresponding to the position on the collection rack of the collection bottle 22a to collect the aforementioned sample, so that when a series of treatments have been completed, the user can easily comprehend the corresponding relationship between each sample on the sample rack 21 and each sample on the collection rack 22 after the treatments.

The sample solution neutralized by the cartridge 17a is sent to the pH meter 18 through the valve 16b. In the case that the pH value of the sample solution is below the preliminarily set threshold value, when the sample solution arrives at the electromagnetic valve 26, the flow channel is switched to the side of the collecting nozzle 24, and the sample solution is collected in the collection bottle 22a. The time for the sample to arrive at the electromagnetic valve 26 is automatically set by the control unit 30, based on the volume flowing through the flow channel and the flow rate of the transported liquid. This arrival time can be also set manually by the user.

On the other hand, in the case that the pH value of the sample solution is above the threshold value, the switching of the flow channel by the electromagnetic valve 26 is not carried out, and the sample which has not been properly neutralized is discharged through a drain. Thereafter, the flow channel switching valves 16a and 16b are switched to the positions represented by the broken line, and the cartridge 17a having attenuated ability for neutralization is regenerated, while neutralization by the cartridge 17b, which has been finished with regeneration, is made possible. For the wasted sample, the sample solution remaining in the sample bottle is taken up by suction by the sampling needle 23, and is subjected again to the sugar chain excision reaction in the reaction tank 15, and then to the neutralization treatment by the cartridge 17b.

When the sugar chain excision reaction, neutralization treatment of a sample, and the collection of the sample solution after treatment are completed, the sampling needle 23 is moved to the above of the sample bottle 21b containing the sample to treat in the next phase, and the above-described operation is repeatedly carried out.

According to the foregoing arrangement, various advantages shown below may be achieved in some implementations. For example, according to the sugar chain excision apparatus of the embodiment of the present invention, a sample solution that has undergone a sugar chain excision reaction using alkali can be neutralized by a simple method, and it is thus possible to perform sugar chain excision stably and efficiently, regardless of the user's experience or level of knowledge.

What is claimed is:

1. A sugar chain excision apparatus for excising sugar chains from complex carbohydrate, comprising:
a sugar chain excising section for mixing a sample solution containing a complex carbohydrate with an alkali solution under heating to excise sugar chains from the complex carbohydrate in the sample solution;
a neutralizing section for neutralizing the sample solution after the excision reaction by the sugar chain excising section, the neutralizing section including a cartridge filled with ion exchange resin; and
a cartridge regenerating section for regenerating the ion exchange resin in the cartridge by passing acid and water through the cartridge.

2. The sugar chain excision apparatus according to claim 1,
wherein the neutralizing section includes a plurality of cartridges filled with ion exchange resin, and
wherein the sugar chain excision apparatus comprises:
a cartridge selecting section for switching between a cartridge to be used for neutralization treatment and a cartridge to be regenerated.

3. The sugar chain excision apparatus according to claim 1, further comprising:
a pH meter provided at the downstream of the neutralizing section; and
an error noticing section for noticing error to the user, when the pH value, as measured with the pH meter, of the sample solution transported from the neutralizing section exceeds a predetermined threshold value.

4. The sugar chain excision apparatus according to claim 3, further comprising:
an error responding section for discarding a sample solution without collecting, when the pH value, as measured with the pH meter, of the sample solution transported from the neutralizing section exceeds a predetermined threshold value, and for subjecting a sample which raises an error again to the excision treatment, after the switching of a cartridge to be used for neutralization by the cartridge selecting section.

5. The sugar chain excision apparatus according to claim 1, further comprising:
a pH meter provided at the downstream of the neutralizing section; and
an error responding section for discarding a sample solution without collecting, when the pH value, as measured with the pH meter, of the sample solution transported from the neutralizing section exceeds a predetermined threshold value, and for subjecting a sample which raises an error again to the excision treatment, after the switching of a cartridge to be used for neutralization by the cartridge selecting section.

6. The sugar chain excision apparatus according to claim 1, further comprising:
a sample introducing/collecting section for introducing a sample solution containing complex carbohydrate to the sugar chain excision apparatus, and for collecting the sample solution after the neutralization by the neutralizing section.
